SEQUENCE LISTING (39 sequences)

SEQ ID NO: 1 - protein, 773 aa, Palaeococcus helgesonii
  MISC_FEATURE (622)..(622): Xaa is Leu, Pro, Gln or Arg
MILDTDYITENGKPVIRIFKKENGEFKIEYDRNFEPYIYALLENEEEIEDIKRITAERHGKKVRIVRAEKVKKKFLGEPIEVWKLVFEHPQDVPDIIRKHPAVVDIYEYDIPFAKRYLIDRGLVPMEGDEELKMLAFDIETFYHEGDEFGEGEILMISYADEGGARVITWKRIDLPYVETVSTEREAIKRFLHVLKEKDPDVLITYNGDNFDFAYIKKRCEKLGLKFTIGRDGSEPKIQRMGDRFAVEVKGIKGRIHLDLYPVVRHTIRLPTYTLEAVYEAVFGKRKEKVYAEEIATAWKSEEGLKRVAQYSMEDAKATYELGREFFPMEVELAKLIGQSVWDVSRSSTGNLVEWYLLREAYERNELAPNKPGDAEYRKRMRSSYLGGYVKEPEKGLWESIAYLDFRSLYPSIIVTHNVSPDTLERECKNYYVAPVVGYRFCSDFKGFIPSILEELIETRQKVKRKMKATIDPVERKMLDYRQRALKILANSYYGYTGYPKARWYSKECAESVTAWGRHYIETTINEAEGFGFKVLYADTDGFFATIPGEKPEVIKKKALEFLKHINKKLPGMLELEYEGFYTRGFFVTKKKYALIDEEGHITTRGLEVVRRDWSEIAKETXAKVLEVILREGSIEKAAGIVKKVVEDLANYRVPVEKLVIHEQITRELKDYKATGPHVAIAKRLQARGIKVKPGTIISYVVLKGSKKISDRVILFDEYDPGRHKYDPDYYIHNQVLPAVLRILEAFGYKEKDLEYQRMRQMGLGAWLGTGKG

SEQ ID NO: 2 - DNA, 2573 bp, Palaeococcus helgesonii
  misc_feature (1865)..(1865): n is a, c, g, or t
atgatacttgatacagattatataacggagaatggaaaacccgttatcaggatttttaagaaggaaaacggcgagtttaaaatagaatacgacaggaattttgagccctacatttacgcgcttctggagaatgaggaggaaatagaggacattaaaaggataaccgccgagaggcacggaaaaaaagtgagaatcgtgcgggctgagaaggttaagaaaaagttcctgggagagcccatagaggtgtggaagcttgtttttgagcatccacaggacgtcccggacattataaggaagcatcctgccgttgtggacatctacgagtacgatatacccttcgcaaagcgctacctcatagacagagggcttgttccgatggagggcgacgaggagctcaaaatgctggcttttgatattgagacgttctaccatgagggagatgaattcggagagggcgaaattttgatgataagctacgccgatgagggcggcgcgagggtgattacgtggaagagaattgacctcccctatgtggaaacggtatccacagagagggaagccataaagcgcttcctccatgttctgaaggaaaaagatccggacgtgctcatcacgtacaacggcgacaacttcgattttgcttacataaaaaagcgctgtgaaaagctcgggttgaagttcacaatcgggagggacggaagcgaaccaaaaattcagaggatgggggatcgcttcgccgtcgaggtcaagggcatcaagggcagaatacaccttgatctctatcccgtcgtgaggcacacaataaggctccccacctatacgcttgaggcggtctatgaagccgttttcggaaagcgaaaggagaaggtctatgcagaagagatagcgacggcatggaagagtgaggaggggcttaagagggtcgcgcagtattcaatggaggatgcaaaagccacatatgagctcggaagggagttcttcccgatggaggtggaactggcaaagctcatagggcagagcgtttgggacgtatcgaggtcaagcacgggcaacctggtggagtggtacctcctgagagaggcatatgagaggaacgagctcgcaccgaataagccgggggatgcggaatacaggaaaagaatgcgctcttcctatctcgggggctacgtcaaggagcccgagaaaggattatgggagagcatagcttatttagattttcgcagcttgtacccctccataatcgtcacccacaacgtttctcccgatacgcttgaaagagaatgcaaaaactattatgtggctccagttgttggctaccgcttctgcagtgactttaagggattcatcccaagcatcctggaggagctcatagaaaccaggcagaaggttaagaggaagatgaaggccacgattgaccccgtggagaggaagatgctcgactacaggcagagggcattgaagattctggcgaatagctattacggttatacgggctatccaaaagcgcgctggtattcgaaggagtgtgccgagagcgtcacggcatgggggaggcactacatagagaccactatcaatgaggcagagggattcgggtttaaagtgctctatgcggacactgatggcttttttgcaacaatacccggtgaaaaaccggaggtcataaaaaagaaggccttggaattcctgaaacacataaataaaaagctccccggaatgctcgagcttgagtatgagggcttctacacgaggggattcttcgtcaccaaaaagaagtacgctctcattgatgaggaggggcacataaccacgaggggccttgaggttgtgaggagggactggagtgagatagcaaaggaaacccnagctaaagtgctggaggtcatcttaagggagggtagcattgaaaaggcagcggggatcgtgaagaaagttgttgaggatctggcaaattaccgcgttcccgtagaaaagctggtcattcacgagcagattacccgggaattaaaggattataaggcgacgggaccccacgtggcgatagcaaagcgccttcaggcaaggggcatcaaggtgaagcccggcaccataataagctatgttgttttgaaggggagcaagaagataagcgacagggtaatcctgttcgatgagtacgaccccggcaggcataagtatgacccagattactacatccacaatcaggttctccccgcggttcttagaatactcgaagccttcggatacaaggagaaagatctggagtaccagaggatgagacagatgggacttggggcgtggcttggaacggggaaggggtgagaggaaatatgccggtaaaagcctcatggaattacttcttccatcctttcgtagattccggcttttctcaaaacctcacggcatgggggaggcactacatagagaccactatcaatgaggcagagggattcgggtttaaagtgctctatgcggacactgatggcttttttgcaacaatacccggtgaaaaaccggaggtcataaaaaagaaggccttggaattcctgaaacacataaataaaaagctcccc

SEQ ID NO: 3 - DNA, 29 bp, Artificial Sequence
  ARCHPOLR1 Primer sequence
cgcgggagaacctggttntcdatrtarta

SEQ ID NO: 4 - protein, 8 aa, Artificial Sequence
  misc_feature (6)..(6): Xaa is Pro or Leu
RGRTWXSX

SEQ ID NO: 5 - DNA, 29 bp, Artificial Sequence
  ARCHPOLF1 Primer sequence
tactacggataggccaargcnagrtggta

SEQ ID NO: 6 - protein, 8 aa, Artificial Sequence
  ARCHPOLF1 Primer translation
YYGAKARW

SEQ ID NO: 7 - DNA, 18 bp, Artificial Sequence
  Primer sequence
tgtaaaacgacggccagt

SEQ ID NO: 8 - DNA, 24 bp, Artificial Sequence
  Primer sequence
agcggataacaatttcacacagga

SEQ ID NO: 9 - DNA, 17 bp, Artificial Sequence
  Primer sequence
catccacaggacgtccc

SEQ ID NO: 10 - protein, 6 aa, Artificial Sequence
  Protein fragment
HPQDVP

SEQ ID NO: 11 - DNA, 20 bp, Artificial Sequence
  Primer sequence
taaacccgaatccctctgcc

SEQ ID NO: 12 - DNA, 20 bp, Artificial Sequence
  Primer sequence
ttgtgtgcctcacgacggga

SEQ ID NO: 13 - DNA, 22 bp, Artificial Sequence
  Primer sequence
gcaaggggcatcaaggtgaagc

SEQ ID NO: 14 - DNA, 22 bp, Artificial Sequence
  Primer sequence
tgttttgaaggggagcaagaag

SEQ ID NO: 15 - DNA, 22 bp, Artificial Sequence
  Primer sequence
gcttttctacgggaacgcggta

SEQ ID NO: 16 - DNA, 20 bp, Artificial Sequence
  Primer sequence
gtgacgctctcggcacactc

SEQ ID NO: 17 - DNA, 20 bp, Artificial Sequence
  Primer sequence
ccacaacggcaggatgcttc

SEQ ID NO: 18 - DNA, 20 bp, Artificial Sequence
  Primer sequence
tagatgtccacaacggcagg

SEQ ID NO: 19 - DNA, 20 bp, Artificial Sequence
  Primer sequence
cagagggcttgttccgatgg

SEQ ID NO: 20 - DNA, 40 bp, Artificial Sequence
  Primer sequence
aagcttccatgggtattcttgatacagattatataacgga

SEQ ID NO: 21 - DNA, 39 bp, Artificial Sequence
  Primer sequence
ggatccgtcgacttaccccttccccgttccaagccacgc

SEQ ID NO: 22 - DNA, 55 bp, Artificial Sequence
  Primer sequence
ttcccctctagaaataattttgtttaactttaagaaggagatatacatatgcacc

SEQ ID NO: 23 - DNA, 55 bp, Artificial Sequence
  Primer sequence
gaattcggatccgctagccatggtatggtgatggtgatggtgcatatgtatatct

SEQ ID NO: 24 - DNA, 24 bp, Artificial Sequence
  Primer sequence
aaattaatacgactcactataggg

SEQ ID NO: 25 - DNA, 19 bp, Artificial Sequence
  Primer sequence
gctagttattgctcagcgg

SEQ ID NO: 26 - DNA, 20 bp, Artificial Sequence
  Primer sequence
cctgctctgccgcttcacgc

SEQ ID NO: 27 - DNA, 24 bp, Artificial Sequence
  Primer sequence
ccatgattcagtgtgcccgtctgg

SEQ ID NO: 28 - protein, 23 aa, Artificial Sequence
  Consensus sequence
EXXXIKXFLXXXXEKDPDXXXTY

SEQ ID NO: 29 - protein, 36 aa, Artificial Sequence
  Consensus sequence
GXVKEPEXGLWXXXXXLDXXXLYPSIIXTHNVSPDT

SEQ ID NO: 30 - protein, 22 aa, Artificial Sequence
  Consensus sequence
GFIPSXLXXLXXXRQXXKXKMK

SEQ ID NO: 31 - protein, 22 aa, Artificial Sequence
  Consensus sequence
DYRQXAXKXLANSXYGYXGYXX

SEQ ID NO: 32 - protein, 7 aa, Artificial Sequence
  Consensus sequence
DTDGXXA

SEQ ID NO: 33 - protein, 23 aa, Artificial Sequence
  Consensus sequence
DEEGXXXTRGLEXVRRDWSXIAK

SEQ ID NO: 34 - protein, 15 aa, Artificial Sequence
  Consensus sequence
LYPSIIXTHNVSPDT

SEQ ID NO: 35 - protein, 16 aa, Artificial Sequence
  Consensus sequence
TRGLEXVRRDWSXIAK

SEQ ID NO: 36 - DNA, 2322 bp, Palaeococcus helgesonii
atgatacttgatacagattatataacggagaatggaaaacccgttatcaggatttttaagaaggaaaacggcgagtttaaaatagaatacgacaggaattttgagccctacatttacgcgcttctggagaatgaggaggaaatagaggacattaaaaggataaccgccgagaggcacggaaaaaaagtgagaatcgtgcgggctgagaaggttaagaaaaagttcctgggagagcccatagaggtgtggaagcttgtttttgagcatccacaggacgtcccggacattataaggaagcatcctgccgttgtggacatctacgagtacgatatacccttcgcaaagcgctacctcatagacagagggcttgttccgatggagggcgacgaggagctcaaaatgctggcttttgatattgagacgttctaccatgagggagatgaattcggagagggcgaaattttgatgataagctacgccgatgagggcggcgcgagggtgattacgtggaagagaattgacctcccctatgtggaaacggtatccacagagagggaagccataaagcgcttcctccatgttctgaaggaaaaagatccggacgtgctcatcacgtacaacggcgacaacttcgattttgcttacataaaaaagcgctgtgaaaagctcgggttgaagttcacaatcgggagggacggaagcgaaccaaaaattcagaggatgggggatcgcttcgccgtcgaggtcaagggcatcaagggcagaatacaccttgatctctatcccgtcgtgaggcacacaataaggctccccacctatacgcttgaggcggtctatgaagccgttttcggaaagcgaaaggagaaggtctatgcagaagagatagcgacggcatggaagagtgaggaggggcttaagagggtcgcgcagtattcaatggaggatgcaaaagccacatatgagctcggaagggagttcttcccgatggaggtggaactggcaaagctcatagggcagagcgtttgggacgtatcgaggtcaagcacgggcaacctggtggagtggtacctcctgagagaggcatatgagaggaacgagctcgcaccgaataagccgggggatgcggaatacaggaaaagaatgcgctcttcctatctcgggggctacgtcaaggagcccgagaaaggattatgggagagcatagcttatttagattttcgcagcttgtacccctccataatcgtcacccacaacgtttctcccgatacgcttgaaagagaatgcaaaaactattatgtggctccagttgttggctaccgcttctgcagtgactttaagggattcatcccaagcatcctggaggagctcatagaaaccaggcagaaggttaagaggaagatgaaggccacgattgaccccgtggagaggaagatgctcgactacaggcagagggcattgaagattctggcgaatagctattacggttatacgggctatccaaaagcgcgctggtattcgaaggagtgtgccgagagcgtcacggcatgggggaggcactacatagagaccactatcaatgaggcagagggattcgggtttaaagtgctctatgcggacactgatggcttttttgcaacaatacccggtgaaaaaccggaggtcataaaaaagaaggccttggaattcctgaaacacataaataaaaagctccccggaatgctcgagcttgagtatgagggcttctacacgaggggattcttcgtcaccaaaaagaagtacgctctcattgatgaggaggggcacataaccacgaggggccttgaggttgtgaggagggactggagtgagatagcaaaggaaacccaagctaaagtgctggaggtcatcttaagggagggtagcattgaaaaggcagcggggatcgtgaagaaagttgttgaggatctggcaaattaccgcgttcccgtagaaaagctggtcattcacgagcagattacccgggaattaaaggattataaggcgacgggaccccacgtggcgatagcaaagcgccttcaggcaaggggcatcaaggtgaagcccggcaccataataagctatgttgttttgaaggggagcaagaagataagcgacagggtaatcctgttcgatgagtacgaccccggcaggcataagtatgacccagattactacatccacaatcaggttctccccgcggttcttagaatactcgaagccttcggatacaaggagaaagatctggagtaccagaggatgagacagatgggacttggggcgtggcttggaacggggaaggggtga

SEQ ID NO: 37 - DNA, 81 bp, Artificial Sequence
  Plasmid fragment
tctagaaataattttgtttaactttaagaaggagatatacatatgcaccatcaccatcaccataccatggctagcggatcc

SEQ ID NO: 38 - protein, 9 aa, Artificial Sequence
  Protein tag sequence
MHHHHHHTM

SEQ ID NO: 39 - protein, 773 aa, Palaeococcus helgesonii
MILDTDYITENGKPVIRIFKKENGEFKIEYDRNFEPYIYALLENEEEIEDIKRITAERHGKKVRIVRAEKVKKKFLGEPIEVWKLVFEHPQDVPDIIRKHPAVVDIYEYDIPFAKRYLIDRGLVPMEGDEELKMLAFDIETFYHEGDEFGEGEILMISYADEGGARVITWKRIDLPYVETVSTEREAIKRFLHVLKEKDPDVLITYNGDNFDFAYIKKRCEKLGLKFTIGRDGSEPKIQRMGDRFAVEVKGIKGRIHLDLYPVVRHTIRLPTYTLEAVYEAVFGKRKEKVYAEEIATAWKSEEGLKRVAQYSMEDAKATYELGREFFPMEVELAKLIGQSVWDVSRSSTGNLVEWYLLREAYERNELAPNKPGDAEYRKRMRSSYLGGYVKEPEKGLWESIAYLDFRSLYPSIIVTHNVSPDTLERECKNYYVAPVVGYRFCSDFKGFIPSILEELIETRQKVKRKMKATIDPVERKMLDYRQRALKILANSYYGYTGYPKARWYSKECAESVTAWGRHYIETTINEAEGFGFKVLYADTDGFFATIPGEKPEVIKKKALEFLKHINKKLPGMLELEYEGFYTRGFFVTKKKYALIDEEGHITTRGLEVVRRDWSEIAKETQAKVLEVILREGSIEKAAGIVKKVVEDLANYRVPVEKLVIHEQITRELKDYKATGPHVAIAKRLQARGIKVKPGTIISYVVLKGSKKISDRVILFDEYDPGRHKYDPDYYIHNQVLPAVLRILEAFGYKEKDLEYQRMRQMGLGAWLGTGKG

(19) United States
(12) Patent Application Publication
(10) Pub. No.: US 2011/0104761 A1
(43) Pub. Date: May 5, 2011

(54) THERMOSTABLE DNA POLYMERASE FROM PALAEOCOCCUS HELGESONII

(75) Inventors: Duncan Clark, Surrey (GB); Nicholas Morant, Surrey (GB)

(73) Assignee: GeneSys Ltd

(21) Appl. No.: 12/922,401

(22) PCT Filed: Mar. 13, 2009
(86) PCT No.: PCT/GB09/50247

§ 371 Date: Sep. 13, 2010

Related U.S. Application Data
(60) Provisional application No. 61/069,429, filed on Mar. 14, 2008.

(30) Foreign Application Priority Data
Mar. 14, 2008 (GB) 0804721.9

(51) Int. Cl. C12P 19/34; C12N 9/12; C12N 15/54; C12N 15/85

(52) U.S. Cl. 435/91.2; 435/194; 536/23.2; 435/320.1; 435/325

(57) ABSTRACT

There is provided a polypeptide having thermostable DNA polymerase activity and comprising or consisting of an amino acid sequence with at least 78% identity to Palaeococcus helgesonii DNA polymerase shown in SEQ ID NO: 1 or SEQ ID NO:39.

### FIELD OF INVENTION

The present invention relates to novel polypeptides having DNA polymerase activity, and their uses.

### BACKGROUND

DNA polymerases are enzymes involved in vivo in DNA repair and replication, but have become an important in vitro diagnostic and analytical tool for the molecular biologist. The enzymes are divided into three main families, based on function and conserved amino acid sequences (see Joyce & Steitz, 1994, Ann. Rev. Biochem. 63: 777-822). In prokaryotes, the main types of DNA polymerases are DNA polymerase I, II and III. DNA polymerase I (encoded by the gene “polA” in E. coli) is considered to be a repair enzyme and has 5′-3′ polymerase activity and often 3′-5′ exonuclease proofreading activity and/or 5′-3′ exonuclease activity which when present mediates nick translation during DNA repair. DNA polymerase II (encoded by the gene “polB” in E. coli) appears to facilitate DNA synthesis starting from a damaged template strand and thus preserves mutations. DNA polymerase III (encoded by the gene “polC” in E. coli) is the replication enzyme of the cell, synthesising nucleotides at a high rate (such as about 30,000 nucleotides per minute) and having no 5′-3′ exonuclease activity.

Other properties of DNA polymerases are derived from their source of origin. For example, several DNA polymerases obtained from thermophilic bacteria have been found to be thermostable, retaining polymerase activity at between 45° C. to 100° C., depending on the polymerase. Thermostable DNA polymerases have found wide use in methods for amplifying nucleic acid sequences by thermocycling amplification reactions such as the polymerase chain reaction (PCR) or by isothermal amplification reactions such as strand displacement amplification (SDA), nucleic acid sequence-based amplification (NASBA), self-sustained sequence replication (3SR), and loop-mediated isothermal amplification (LAMP).

The different properties of thermostable DNA polymerases, such as level of thermostability, strand displacement activity, fidelity (error rate) and binding affinity to template DNA and/or RNA and/or free nucleotides, make them suited to different types of amplification reaction. For example, thermostable (typically at temperatures up to 94° C.), high-fidelity (typically with 3′-5′ exonuclease proof-reading activity), processive and rapidly synthesising DNA polymerases are preferred for PCR. Enzymes which do not discriminate significantly between dideoxy and deoxy nucleotides may be preferred for sequencing. Meanwhile, isothermal amplification reactions require a DNA polymerase with strong strand displacement activity.

The proof-reading DNA polymerases currently available commercially for PCR are derived from species within either the Pyrococcus genus or the Thermococcus genus of hyperthermophilic euryarchaeota. Archaea are a third domain of living organisms, distinct from Bacteria and Eucarya. These organisms have been isolated predominantly from deep-sea hydrothermal vents (“black smokers”) and typically have optimal growth temperatures around 85-99° C. Examples of key species from which proof-reading DNA polymerases for use in PCR have been isolated include Thermococcus barossii, Thermococcus litoralis, Thermococcus gorgonarius, Thermococcus pacificus, Thermococcus zilligii, Thermococcus 9N7, Thermococcus fumicolans, Thermococcus aggregans (TY), Thermococcus peptonophilus, Pyrococcus furiosus, Pyrococcus sp. and Thermococcus KOD.

Takagi et al. (Appl. Env. Microbiol. (1997) 63: 4504-4510) and EP-A-0745675 provide characterisation of the DNA polymerase found in Pyrococcus sp. Strain KOD1. This strain has an optimum growth temperature of 95° C. U.S. Pat. No. 7,045,328 discloses a DNA polymerase from P. furiosus and U.S. Pat. No. 5,834,285 a DNA polymerase from T. litoralis. Griffiths et al. (Prot. Exp. and Purification (2006) 52 19-30) discloses polymerases from Thermococcus species T. ziglligii and Thermococcus ‘GT’.

### SUMMARY OF INVENTION

The present invention provides in one aspect a novel thermostable DNA polymerase for use in reactions requiring DNA polymerase activity such as nucleic acid amplification reactions. The polymerase has been isolated from a new genus of hyperthermophilic euryarchaeota, the Palaeococcus genus, which represents a deep-branching lineage of the order Thermococcales that diverged before Thermococcus and Pyrococcus. Surprisingly, the polymerase is suitable for use in thermocycling amplification reactions, even though the optimum growth temperature for the organism is only 80° C. (see below).

According to one aspect of the present invention there is provided a polypeptide having thermostable DNA polymerase activity and comprising or consisting essentially of an amino acid sequence with at least 78% identity, for example at least 80%, 85%, 90% or 95% identity, to Palaeococcus helgesonii DNA polymerase shown in SEQ ID NO: 1. The polypeptide may, for example, have 78%, 79%, 81%, 82%, 83%, 84%, 86%, 87%, 88%, 89% 91%, 92%, 93%, 94%, 96%, 97%, 98% or even 99% identity to SEQ ID NO: 1. Preferably, the polypeptide is isolated.

The P. helgesonii DNA polymerase has the following amino acid sequence:

(SEQ ID NO: 1)
MILDTDYITENGKPVIRIFKKENGEFKIEYDRNFEPYIYALLENEEEIE
DIKRITAERHGKKVRIVRAEKVKKKFLGEPIEVWKLVFEHPQDVPDIIR
KHPAVVDIYEYDIPFAKRYLIDRGLVPMEGDEELKMLAFDIETFYHEGD
EFGEGEILMISYADEGGARVITWKRIDLPYVETVSTEREAIKRFLHVLK
EKDPDVLITYNGDNFDFAYIKKRCEKLGLKFTIGRDGSEPKIQRMGDRF
AVEVKGIKGRIHLDLYPVVRHTIRLPTYTLEAVYEAVFGKRKEKVYAEE
IATAWKSEEGLKRVAQYSMEDAKATYELGREFFPMEVELAKLIGQSVWD
VSRSSTGNLVEWYLLREAYERNELAPNKPGDAEYRKRMRSSYLGGYVKE
PEKGLWESIAYLDFRSLYPSIIVTHNVSPDTLERECKNYYVAPVVGYRF
CSDFKGFIPSILEELIETRQKVKRKMKATIDPVERKMLDYRQRALKILA
NSYYGYTGYPKARWYSKECAESVTAWGRHYIETTINEAEGFGFKVLYAD
TDGFFATIPGEKPEVIKKKALEFLKHINKKLPGMLELEYEGFYTRGFFV
TKKKYALIDEEGHITTRGLEVVRRDWSEIAKETXAKVLEVILREGSIEK
AAGIVKKVVEDLANYRVPVEKLVIHEQITRELKDYKATGPHVAIAKRLQ
ARGIKVKPGTIISYVVLKGSKKISDRVILFDEYDPGRHKYDPDYYIHNQ
VLPAVLRILEAFGYKEKDLEYQRMRQMGLGAWLGTGKG.

The underlined amino acid “X” has been confirmed as being “Q” and, therefore, a preferred embodiment of the polypeptide according to the invention has the amino acid sequence:

(SEQ ID NO: 39)
MILDTDYITENGKPVIRIFKKENGEFKIEYDRNFEPYIYALLENEEEIE
DIKRITAERHGKKVRIVRAEKVKKKFLGEPIEVWKLVFEHPQDVPDIIR
KHPAVVDIYEYDIPFAKRYLIDRGLVPMEGDEELKMLAFDIETFYHEGD
EFGEGEILMISYADEGGARVITWKRIDLPYVETVSTEREAIKRFLHVLK
EKDPDVLITYNGDNFDFAYIKKRCEKLGLKFTIGRDGSEPKIQRMGDRF
AVEVKGIKGRIHLDLYPVVRHTIRLPTYTLEAVYEAVFGKRKEKVYAEE
IATAWKSEEGLKRVAQYSMEDAKATYELGREFFPMEVELAKLIGQSVWD
VSRSSTGNLVEWYLLREAYERNELAPNKPGDAEYRKRMRSSYLGGYVKE
PEKGLWESIAYLDFRSLYPSIIVTHNVSPDTLERECKNYYVAPVVGYRF
CSDFKGFIPSILEELIETRQKVKRKMKATIDPVERKMLDYRQRALKILA
NSYYGYTGYPKARWYSKECAESVTAWGRHYIETTINEAEGFGFKVLYAD
TDGFFATIPGEKPEVIKKKALEFLKHINKKLPGMLELEYEGFYTRGFFV
TKKKYALIDEEGHITTRGLEVVRRDWSEIAKETQAKVLEVILREGSIEK
AAGIVKKVVEDLANYRVPVEKLVIHEQITRELKDYKATGPHVAIAKRLQ
ARGIKVKPGTIISYVVLKGSKKISDRVILFDEYDPGRHKYDPDYYIHNQ
VLPAVLRILEAFGYKEKDLEYQRMRQMGLGAWLGTGKG.

Preferably, the polypeptide has thermostable DNA polymerase activity and comprises or consists essentially of an amino acid sequence with at least 78% identity, for example at least 80%, 85%, 90% or 95% identity, to Palaeococcus helgesonii DNA polymerase shown in SEQ ID NO: 39. The polypeptide may, for example, have 78%, 79%, 81%, 82%, 83%, 84%, 86%, 87%, 88%, 89% 91%, 92%, 93%, 94%, 96%, 97%, 98% or even 99% identity to SEQ ID NO: 39. Preferably, the polypeptide is isolated.

The predicted molecular weight of this 773 amino acid residue P. helgesonii DNA polymerase shown in SEQ ID NO: 39 is about 89,750 Daltons.

The above percentage sequence identity may be determined using the BLASTP computer program with SEQ ID NO:1 or 39 as the base sequence. This means that SEQ ID NO:1 or 39 is the sequence against which the percentage identity is determined. The BLAST software is publicly available at http://blast.ncbi.nlm.nih.gov/Blast.cgi (accessible on 12 Mar. 2009).

For example, the polypeptide may comprise or consist essentially of any contiguous 603 amino acid sequence included within SEQ ID NO:39. For example, the polypeptide may comprise from about 580 to 773, about 600 to 750 or about 650 to 700 contiguous amino acids included within SEQ ID NO:39.

The polypeptide may comprise or consist essentially of the amino acid sequence SEQ ID NO:39, or of the amino acid sequence of SEQ ID NO:39 with 1, 2, 3, 4, 5, 6, 7, 8, 9, 10, 11, 12, 13, 14, 15, or about 20 amino acids or contiguous amino acids added to or removed from any part of the polypeptide and/or 1, 2, 3, 4, 5, 6, 7, 8, 9, 10, 11, 12, 13, 14, 15 or about 20 amino acids or contiguous amino acids added to or removed from the N-terminus region and/or the C-terminus region.

Palaeococcus helgesonii is a facultatively anaerobic hyperthermophilic archaeon isolated from a shallow geothermal well in the southern Tyrrhenian Sea, Italy, and has a reported temperature range for growth of 45-85° C. and an optimum growth temperature of about 80° C. (see Amend et al., 2003, Arch. Microbiol. 179: 394-401). This organism was reported to be the second member of the Palaeococcus genus of hyperthermophilic euryarchaeota, and to date there are no known published reports of the identification and characterisation of a DNA polymerase from this genus. Genomic DNA (gDNA) from P. helgesonii has been isolated by the inventors who used a sophisticated gene walking technique to clone a DNA polymerase, considered to be a DNA polymerase II encoded by a DNA polymerase II (polB) gene.

DNA polymerase II enzymes comprise certain conserved motifs, for example, as described in Kim et al., (2007) J. Microbiol. Biotechnol. 17 1090-1097. Therefore, in a preferred embodiment, the peptide according to the invention comprises one or more of the amino acid sequences:

(SEQ ID NO: 28)
EX₁X₂X₁₈IKX₃FLX₁₉X₄X₂₀X₁EKDPDX₄X₅X₄TY
(SEQ ID NO: 29)
GX₆VKEPEX₁GLWX₂X₂₁X₅X₂₂X₈LDX₆X₁X₉LYPSIIX₄THNVSPDT
(SEQ ID NO: 30)
GFIPSX₅LX₁₀X₁₁L X₅X₂X₂₃RQX₁₂X₄KX₁₃KMK
(SEQ ID NO: 31)
DYRQX₁AX₅KX₅LANSX₆YGYX₂₄GYX₁₄X₁
(SEQ ID NO: 32)
DTDGX₁₅X₁₆A
(SEQ ID NO: 33)
DEEGX₂₅X₄X₁₇TRGLEX₄VRRDWSX₂IAK

where:
X₁=K or R X₁₄=A or P
X₂=E or D X₁₅=F or L
X₃=R or A X₁₆=Y, F or H
X₄=V or I X₁₇=V, T or I
X₅=L or I X₁₈=M or A
X₆=Y or F X₁₉=K, R or H
X₇=N or G X₂₀=V, I or L
X₈=Y or S X₂₁=N, G or S
X₁₀=G, K or E X₂₃=E or T
X₁₁=N, D, H or E X₂₄=Y or T
X₁₂=K or E X₂₅=G or H
X₁₃=R, K or T

For example, the polypeptide may comprise any two, any three, any four or any five amino acid sequences selected from SEQ ID NOs: 28-33 or may comprise all of amino acid sequences SEQ ID NOs: 28-33. In a preferred embodiment, the peptide according to the invention may comprise one or both of the amino acid sequences:

| | |
|---|---|
| LYPSIIX₄THNVSPDT | (SEQ ID NO: 34) |
| TRGLEX₄VRRDWSX₂IAK. | (SEQ ID NO: 35) |

The polypeptide may be suitable for carrying out a thermocycling amplification reaction, such as a polymerase chain reaction (PCR). This characteristic requires sufficient thermostability to withstand the denaturation cycle, normally 95° C.

The polypeptide of the invention may be sufficiently stable to allow it to be functional in a thermocycling reaction such as PCR (for example, as exemplified in Example 8 below). Even though P. helgesonii has a reported growth range of up to 88° C. (see above), the inventors have surprisingly found that even a crude extract of the DNA polymerase II of SEQ ID NO:39 is sufficiently stable for use in PCR.

The polypeptide may have 3′-5′ exonuclease proofreading activity.

In some embodiments, the polypeptide may lack 5′-3′ exonuclease activity.

The polypeptide of the invention may be an isolated thermostable DNA polymerase obtainable from Palaeococcus helgesonii and having a molecular weight of about 90,000 Daltons, or about 89,000-about 91,000 Daltons, or an enzymatically active fragment thereof. The term “enzymatically active fragment” means a fragment of such a polymerase obtainable from P. helgesonii and having enzyme activity which is at least 60%, preferably at least 70%, more preferably at least 80%, yet more preferably 90%, 95%, 96%, 97%, 98%, 99% or 100% that of the full length polymerase being compared to. The given activity may be determined by any standard measure, for example, the number of bases of nucleotides of the template sequence which can be replicated in a given time period. The skilled person is routinely able to determine such properties and activities.

The polypeptide of the invention may be suitable for use in one or more reactions requiring DNA polymerase activity, for example one or more of the group consisting of: nick translation, second-strand cDNA synthesis in cDNA cloning, DNA sequencing, and thermocycling amplification reactions such as PCR.

In a further aspect of the invention the polypeptide exhibits high fidelity polymerase activity during a thermocycling amplification reaction (such as PCR). High fidelity may be defined as a PCR error rate of less than 1 nucleotide per 300×10⁶ amplified nucleotides, for example less than 1 nucleotide per 250×10⁶, 200×10⁶, 150×10⁶, 100×10⁶ or 50×10⁶ amplified nucleotides. Alternatively, the error rate of the polypeptides may be in the range 1-300 nucleotides per 10⁶ amplified nucleotides, for example 1-200, 1-100, 100-300, 200-300, 100-200 or 75-200 nucleotides per 10⁶ amplified nucleotides. Error rate may be determined using the opal reversion assay as described by Kunkel et al. (1987, Proc. Natl. Acad. Sci. USA 84: 4865-4869).

The polypeptide of the invention may comprise additional functional and structural domains, for example, an affinity purification tag (such as an His purification tag), or DNA polymerase activity-enhancing domains such as the proliferating cell nuclear antigen homologue from Archaeoglobus fulgidus, T3 DNA polymerase thioredoxin binding domain, DNA binding protein Sso7d from Sulfolobus solfataricus, Sso7d-like proteins, or mutants thereof, or helix-hairpin-helix motifs derived from DNA topoisomerase V. The DNA polymerase activity-enhancing domain may also be a Cren7 enhancer domain or variant thereof, as defined and exemplified in co-pending International patent application no. PCT/GB2009/000063, which discloses that this highly conserved protein domain from Crenarchael organisms is useful to enhance the properties of a DNA polymerase. International patent application no. PCT/GB2009/000063 is incorporated herein by reference in its entirety.

In another aspect of the invention there is provided a composition comprising the polypeptide as described herein. The composition may for example include a buffer, and/or most or all ingredients for performing a reaction (such as a DNA amplification reaction for example PCR), and/or a stabiliser (such as E. coli GroEL protein, to enhance thermostability), and/or other compounds. The composition is in one aspect enzymatically thermostable.

The invention further provides an isolated nucleic acid encoding the polypeptide with identity to P. helgesonii DNA polymerase. The nucleic acid may, for example, have a sequence as shown below (5′-3′):

(SEQ ID NO: 2)
atgatacttgatacagattatataacggagaatggaaaacccgttatc
aggatttttaagaaggaaaacggcgagtttaaaatagaatacgacagg
aattttgagccctacatttacgcgcttctggagaatgaggaggaaata
gaggacattaaaaggataaccgccgagaggcacggaaaaaaagtgaga
atcgtgcgggctgagaaggttaagaaaaagttcctgggagagcccata
gaggtgtggaagcttgtttttgagcatccacaggacgtcccggacatt
ataaggaagcatcctgccgttgtggacatctacgagtacgatataccc
ttcgcaaagcgctacctcatagacagagggcttgttccgatggagggc
gacgaggagctcaaaatgctggcttttgatattgagacgttctaccat
gagggagatgaattcggagagggcgaaattttgatgataagctacgcc
gatgagggcggcgcgagggtgattacgtggaagagaattgacctcccc
tatgtggaaacggtatccacagagagggaagccataaagcgcttcctc
catgttctgaaggaaaaagatccggacgtgctcatcacgtacaacggc
gacaacttcgattttgcttacataaaaaagcgctgtgaaaagctcggg
ttgaagttcacaatcgggagggacggaagcgaaccaaaaattcagagg
atgggggatcgcttcgccgtcgaggtcaagggcatcaagggcagaata
caccttgatctctatcccgtcgtgaggcacacaataaggctccccacc
tatacgcttgaggcggtctatgaagccgttttcggaaagcgaaaggag
aaggtctatgcagaagagatagcgacggcatggaagagtgaggagggg
cttaagagggtcgcgcagtattcaatggaggatgcaaaagccacatat
gagctcggaagggagttcttcccgatggaggtggaactggcaaagctc
atagggcagagcgtttgggacgtatcgaggtcaagcacgggcaacctg
gtggagtggtacctcctgagagaggcatatgagaggaacgagctcgca
ccgaataagccgggggatgcggaatacaggaaaagaatgcgctcttcc
tatctcgggggctacgtcaaggagcccgagaaaggattatgggagagc
atagcttatttagattttcgcagcttgtaccectccataatcgtcacc
cacaacgtttctcccgatacgcttgaaagagaatgcaaaaactattat
gtggctccagttgttggctaccgcttctgcagtgactttaagggattc
atcccaagcatcctggaggagctcatagaaaccaggcagaaggttaag
aggaagatgaaggccacgattgaccccgtggagaggaagatgctcgac
tacaggcagagggcattgaagattctggcgaatagctattacggttat
acgggctatccaaaagcgcgctggtattcgaaggagtgtgccgagagc
gtcacggcatgggggaggcactacatagagaccactatcaatgaggca
gagggattcgggtttaaagtgctctatgcggacactgatggctttttt
gcaacaatacccggtgaaaaaccggaggtcataaaaaagaaggccttg
gaattcctgaaacacataaataaaaagctccccggaatgctcgagctt
gagtatgagggcttctacacgaggggattcttcgtcaccaaaaagaag
tacgctctcattgatgaggaggggcacataaccacgaggggccttgag
gttgtgaggagggactggagtgagatagcaaaggaaacccNagctaaa
gtgctggaggtcatcttaagggagggtagcattgaaaaggcagcgggg
atcgtgaagaaagttgttgaggatctggcaaattaccgcgttcccgta
gaaaagctggtcattcacgagcagattacccgggaattaaaggattat
aaggcgacgggaccccacgtggcgatagcaaagcgccttcaggcaagg
ggcatcaaggtgaagcccggcaccataataagctatgttgttttgaag
gggagcaagaagataagcgacagggtaatcctgttcgatgagtacgac
cccggcaggcataagtatgacccagattactacatccacaatcaggtt
ctccccgcggttcttagaatactcgaagccttcggatacaaggagaaa
gatctggagtaccagaggatgagacagatgggacttggggcgtggctt
ggaacggggaaggggtgagaggaaatatgccggtaaaagcctcatgga
attacttatccatcctttcgtagattccggctttctcaaaacctcacg
gcatgggggaggcactatagagaccactatcaatgaggcagagggatt
cgggtttaaagtgctctatgcggacactgatggcttttttgcaacaat
acccggtgaaaaaccggaggtcataaaaaagaaggccttggaattcct
tgaaacacataaataaaaagctcccc.

The non-italic underlined sequence above is outside the polymerase gene sequence and the capitalised nucleic acid “N” has been confirmed as being “A”, so in a preferred embodiment the nucleic acid has the sequence shown below (5′-3′):

(SEQ ID NO: 36)
atgatacttgatacagattatataacggagaatggaaaacccgttatc
aggatttttaagaaggaaaacggcgagtttaaaatagaatacgacagg
aattttgagccctacatttacgcgcttctggagaatgaggaggaaata
gaggacattaaaaggataaccgccgagaggcacggaaaaaaagtgaga
atcgtgcgggctgagaaggttaagaaaaagttcctgggagagcccata
gaggtgtggaagcttgtttttgagcatccacaggacgtcccggacatt
ataaggaagcatcctgccgttgtggacatctacgagtacgatataccc
ttcgcaaagcgctacctcatagacagagggcttgttccgatggagggc
gacgaggagctcaaaatgctggcttttgatattgagacgttctaccat
gagggagatgaattcggagagggcgaaattttgatgataagctacgcc
gatgagggcggcgcgagggtgattacgtggaagagaattgacctcccc
tatgtggaaacggtatccacagagagggaagccataaagcgcttcctc
catgttctgaaggaaaaagatccggacgtgctcatcacgtacaacggc
gacaacttcgattttgcttacataaaaaagcgctgtgaaaagctcggg
ttgaagttcacaatcgggagggacggaagcgaaccaaaaattcagagg
atgggggatcgcttcgccgtcgaggtcaagggcatcaagggcagaata
caccttgatctctatcccgtcgtgaggcacacaataaggctccccacc
tatacgcttgaggcggtctatgaagccgttttcggaaagcgaaaggag
aaggtctatgcagaagagatagcgacggcatggaagagtgaggagggg
cttaagagggtcgcgcagtattcaatggaggatgcaaaagccacatat
gagctcggaagggagttcttcccgatggaggtggaactggcaaagctc
atagggcagagcgtttgggacgtatcgaggtcaagcacgggcaacctg
gtggagtggtacctcctgagagaggcatatgagaggaacgagctcgca
ccgaataagccgggggatgcggaatacaggaaaagaatgcgctcttcc
tatctcgggggctacgtcaaggagcccgagaaaggattatgggagagc
atagcttatttagattttcgcagcttgtaccectccataatcgtcacc
cacaacgtttctcccgatacgcttgaaagagaatgcaaaaactattat
gtggctccagttgttggctaccgcttctgcagtgactttaagggattc
atcccaagcatcctggaggagctcatagaaaccaggcagaaggttaag
aggaagatgaaggccacgattgaccccgtggagaggaagatgctcgac
tacaggcagagggcattgaagattctggcgaatagctattacggttat
acgggctatccaaaagcgcgctggtattcgaaggagtgtgccgagagc
gtcacggcatgggggaggcactacatagagaccactatcaatgaggca
gagggattcgggtttaaagtgctctatgcggacactgatggctttttt
gcaacaatacccggtgaaaaaccggaggtcataaaaaagaaggccttg
gaattcctgaaacacataaataaaaagctccccggaatgctcgagctt
gagtatgagggcttctacacgaggggattcttcgtcaccaaaaagaag
tacgctctcattgatgaggaggggcacataaccacgaggggccttgag
gttgtgaggagggactggagtgagatagcaaaggaaacccaagctaaa
gtgctggaggtcatcttaagggagggtagcattgaaaaggcagcgggg
atcgtgaagaaagttgttgaggatctggcaaattaccgcgttcccgta
gaaaagctggtcattcacgagcagattacccgggaattaaaggattat
aaggcgacgggaccccacgtggcgatagcaaagcgccttcaggcaagg
ggcatcaaggtgaagcccggcaccataataagctatgttgttttgaag
gggagcaagaagataagcgacagggtaatcctgttcgatgagtacgac
cccggcaggcataagtatgacccagattactacatccacaatcaggtt
ctccccgcggttcttagaatactcgaagccttcggatacaaggagaaa
gatctggagtaccagaggatgagacagatgggacttggggcgtggctt
ggaacggggaaggggtga

The nucleotide of SEQ ID NO: 36 encodes the P. helgesonii DNA polymerase of SEQ ID NO:39 as follows:

| | |
|---|---|
| 1 atgatacttgatacagattatataacggagaatggaaaacccgttatcaggatttttaag | (SEQ ID NO: 36) |
| 1 M I L D T D Y I T E N G K P V I R I F K | (SEQ ID NO: 39) |
| 61 aaggaaaacggcgagtttaaaatagaatacgacaggaattttgagccctacatttacgcg | |
| 21 K E N G E F K I E Y D R N F E P Y I Y A | |
| 121 cttctggagaatgaggaggaaatagaggacattaaaaggataaccgccgagaggcacgga | |
| 41 L L E N E E E I E D I K R I T A E R H G | |
| 181 aaaaaagtgagaatcgtgcgggctgagaaggttaagaaaaagttcctgggagagcccata | |
| 61 K K V R I V R A E K V K K K F L G E P I | |
| 241 gaggtgtggaagcttgtttttgagcatccacaggacgtcccggacattataaggaagcat | |
| 81 E V W K L V F E H P Q D V P D I I R K H | |
| 301 cctgccgttgtggacatctacgagtacgatatacccttcgcaaagcgctacctcatagac | |
| 101 P A V V D I Y E Y D I P F A K R Y L I D | |
| 361 agagggcttgttccgatggagggcgacgaggagctcaaaatgctggcttttgatattgag | |
| 121 R G L V P M E G D E E L K M L A F D I E | |
| 421 acgttctaccatgagggagatgaattcggagagggcgaaattttgatgataagctacgcc | |
| 141 T F Y H E G D E F G E G E I L M I S Y A | |
| 481 gatgagggcggcgcgagggtgattacgtggaagagaattgacctcccctatgtggaaacg | |
| 161 D E G G A R V I T W K R I D L P Y V E T | |
| 541 gtatccacagagagggaagccataaagcgcttcctccatgttctgaaggaaaaagatccg | |
| 181 V S T E R E A I K R E L H V L K E K D P | |
| 601 gacgtgctcatcacgtacaacggcgacaacttcgattttgcttacataaaaaagcgctgt | |
| 201 D V L I T Y N G D N E D F A Y I K K R C | |
| 661 gaaaagctcgggttgaagttcacaatcgggagggacggaagcgaaccaaaaattcagagg | |
| 221 E K L G L K F T I G R D G S E P K I Q R | |
| 721 atgggggatcgcttcgccgtcgaggtcaagggcatcaagggcagaatacaccttgatctc | |
| 241 M G D R F A V E V K G I K G R I H L D L | |
| 781 tatcccgtcgtgaggcacacaataaggctccccacctatacgcttgaggcggtctatgaa | |
| 261 Y P V V R H T I R L P T Y T L E A V Y E | |
| 841 gccgttttcggaaagcgaaaggagaaggtctatgcagaagagatagcgacggcatggaag | |
| 281 A V E G K R K E K V Y A E E I A T A W K | |
| 901 agtgaggaggggcttaagagggtcgcgcagtattcaatggaggatgcaaaagccacatat | |
| 301 S E E G L K R V A Q Y S M E D A K A T Y | |
| 961 gagctcggaagggagttcttcccgatggaggtggaactggcaaagctcatagggcagagc | |
| 321 E L G R E F F P M E V E L A K L I G Q S | |
| 1021 gtttgggacgtatcgaggtcaagcacgggcaacctggtggagtggtacctcctgagagag | |
| 341 V W D V S R S S I G N L V E W Y L L R E | |
| 1081 gcatatgagaggaacgagctcgcaccgaataagccgggggatgcggaatacaggaaaaga | |
| 361 A Y E R N E L A P N K P G D A E Y R K R | |
| 1141 atgcgctcttcctatctcgggggctacgtcaaggagcccgagaaaggattatgggagagc | |
| 381 M R S S Y L G G Y V K E P E K G L W E S | |
| 1201 atagcttatttagattttcgcagcttgtacccctccataatcgtcacccacaacgtttct | |
| 401 I A Y L D E R S L Y P S I I V I H N V S | |
| 1261 cccgatacgcttgaaagagaatgcaaaaactattatgtggctccagttgttggctaccgc | |
| 421 P D T L E R E C K N Y Y V A P V V G Y R | |
| 1321 ttctgcagtgactttaagggattcatcccaagcatcctggaggagctcatagaaaccagg | |
| 441 F C S D F K G F I P S I L E E L I E T R | |
| 1381 cagaaggttaagaggaagatgaaggccacgattgaccccgtggagaggaagatgctcgac | |
| 461 Q K V K R K M K A T I D P V E R K M L D | |
| 1441 tacaggcagagggcattgaagattctggcgaatagctattacggttatacgggctatcca | |
| 481 Y R Q R A L K I L A N S Y Y G Y T G Y P | |
| 1501 aaagcgcgctggtattcgaaggagtgtgccgagagcgtcacggcatgggggaggcactac | |
| 501 K A R W Y S K E C A E S V T A W G R H Y | |
| 1561 atagagaccactatcaatgaggcagagggattcgggtttaaagtgctctatgcggacact | |
| 521 I E T T I N E A E G E G F K V L Y A D T | |
| 1621 gatggcttttttgcaacaatacccggtgaaaaaccggaggtcataaaaaagaaggccttg | |
| 541 D G E E A T I P G E K P E V I K K K A L | |
| 1681 gaattcctgaaacacataaataaaaagctccccggaatgctcgagcttgagtatgagggc | |
| 561 E F L K H I N K K L P G M L E L E Y E G | |
| 1741 ttctacacgaggggattcttcgtcaccaaaaagaagtacgctctcattgatgaggagggg | |
| 581 F Y T R G E F V T K K K Y A L I D E E G | |
| 1801 cacataaccacgaggggccttgaggttgtgaggagggactggagtgagatagcaaaggaa | |
| 601 H I T T R G L E V V R R D W S E I A K E | |
| 1861 acccaagctaaagtgctggaggtcatcttaagggagggtagcattgaaaaggcagcgggg | |
| 621 T Q A K V L E V I L R E G S I E K A A G | |
| 1921 atcgtgaagaaagttgttgaggatctggcaaattaccgcgttcccgtagaaaagctggtc | |
| 641 I V K K V V E D L A N Y R V P V E K L V | |
| 1981 attcacgagcagattacccgggaattaaaggattataaggcgacgggaccccacgtggcg | |
| 661 I H E Q I T R E L K D Y K A T G P H V A | |
| 2041 atagcaaagcgccttcaggcaaggggcatcaaggtgaagcccggcaccataataagctat | |
| 681 I A K R L Q A R G I K V K P G T I I S Y | |
| 2101 gttgttttgaaggggagcaagaagataagcgacagggtaatcctgttcgatgagtacgac | |
| 701 V V L K G S K K I S D R V I L F D E Y D | |
| 2161 cccggcaggcataagtatgacccagattactacatccacaatcaggttctccccgcggtt | |
| 721 P G R H K Y D P D Y Y I H N Q V L P A V | |
| 2221 cttagaatactcgaagccttcggatacaaggagaaagatctggagtaccagaggatgaga | |
| 741 L R I L E A F G Y K E K D L E Y Q R M R | |
| 2281 cagatgggacttggggcgtggcttggaacggggaaggggtga | |
| 761 Q M G L G A W L G T G K G *. | |

The underlined and italicised codon “ata” coding for Isoleucine in SEQ ID NOs:2 & 36 above is a minor tRNA in E. coli and, therefore, this codon was changed to “att” by the inventors for expression clone work (see Henaut and Danchin (1996) in Escherichia coli and Salmonella typhimurium Cellular and Molecular Biology Vol. 2, 2047-2066, American Society for Microbiology, Washington, D.C.). The isolated nucleic acid having this amended nucleotide sequence is also encompassed by the invention. The altered codon does not result in any change in the expressed amino acid sequence which is also, therefore, SEQ ID NO:39.

In addition, as described in the Examples below, a “gga” motif (encoding for Glycine) was added by the inventors after the first three bases of SEQ ID NOs:2 & 36, so the first nine bases were “atgggaatt”. The isolated nucleic acid variant of SEQ ID NOs:2 & 36, incorporating these changes, is encompassed by the invention, as is the isolated protein having the amino acid sequence encoded by the variants. The “gga” codon was added to introduce an NcoI restriction enzyme recognition sequence.

Also encompassed by the invention are further variants of the nucleic acids, as defined below.

Further provided is a vector comprising the isolated nucleic acid as described herein.

Additionally provided is a host cell transformed with the nucleic acid or the vector of the invention.

Also provided is a method for of producing a DNA polymerase of the invention comprising culturing the host cell defined herein under conditions suitable for expression of the DNA polymerase.

A recombinant polypeptide expressed from the host cell is also encompassed by the invention.

In another aspect of the invention there is provided a kit comprising the polypeptide as described herein, and/or the composition as described herein, and/or the isolated nucleic acid as described herein, and/or the vector as described herein, and/or the host cell as described herein, together with packaging materials therefor. The kit may, for example, comprise components including the polypeptide for carrying out a reaction requiring DNA polymerase activity, such as PCR.

The invention further provides a method of amplifying a sequence of a target nucleic acid using a thermocycling reaction, for example PCR, comprising the steps of:

(1) contacting the target nucleic acid with the polypeptide having thermostable DNA polymerase activity or the composition as described herein; and
(2) incubating the target nucleic acid with the polypeptide or the composition under thermocycling reaction conditions which allow amplification of the target nucleic acid.

The present invention also encompasses variants of the polypeptide as defined herein. As used herein, a “variant” means a polypeptide in which the amino acid sequence differs from the base sequence from which it is derived in that one or more amino acids within the sequence are substituted for other amino acids. Amino acid substitutions may be regarded as “conservative” where an amino acid is replaced with a different amino acid with broadly similar properties. Non-conservative substitutions are where amino acids are replaced with amino acids of a different type.

By “conservative substitution” is meant the substitution of an amino acid by another amino acid of the same class, in which the classes are defined as follows:

| Class | Amino acid examples |
|---|---|
| Nonpolar: | A, V, L, I, P, M, F, W |
| Uncharged polar: | G, S, T, C, Y, N, Q |
| Acidic: | D, E |
| Basic: | K, R, H. |

As is well known to those skilled in the art, altering the primary structure of a peptide by a conservative substitution may not significantly alter the activity of that peptide because the side-chain of the amino acid which is inserted into the sequence may be able to form similar bonds and contacts as the side chain of the amino acid which has been substituted out. This is so even when the substitution is in a region which is critical in determining the peptide's conformation.

Non-conservative substitutions are possible provided that these do not interrupt with the function of the DNA binding domain polypeptides.

Broadly speaking, fewer non-conservative substitutions will be possible without altering the biological activity of the polypeptides.

Determination of the effect of any substitution (and, indeed, of any amino acid deletion or insertion) is wholly within the routine capabilities of the skilled person, who can readily determine whether a variant polypeptide retains the thermostable DNA polymerase activity according to the invention. For example, when determining whether a variant of the polypeptide falls within the scope of the invention, the skilled person will determine whether the variant retains enzyme activity (i.e., polymerase activity) at least 60%, preferably at least 70%, more preferably at least 80%, yet more preferably 90%, 95%, 96%, 97%, 98%, 99% or 100% of the non-variant polypeptide. Activity may be measured by, for example, any standard measure such as the number of bases of a template sequence which can be replicated in a given time period.

Variants of the polypeptide may comprise or consist essentially of an amino acid sequence with at least 78% identity, for example at least 79%, 81%, 82%, 83%, 84%, 86%, 87%, 88%, 89% 91%, 92%, 93%, 94%, 96%, 97%, 98% or 99% identity to SEQ ID NO:1.

Using the standard genetic code, further nucleic acids encoding the polypeptides may readily be conceived and manufactured by the skilled person. The nucleic acid may be DNA or RNA and, where it is a DNA molecule, it may for example comprise a cDNA or genomic DNA.

The invention encompasses variant nucleic acids encoding the polypeptide of the invention. The term “variant” in relation to a nucleic acid sequences means any substitution of, variation of, modification of, replacement of deletion of, or addition of one or more nucleic acid(s) from or to a polynucleotide sequence providing the resultant polypeptide sequence encoded by the polynucleotide exhibits at least the same properties as the polypeptide encoded by the basic sequence. The term therefore includes allelic variants and also includes a polynucleotide which substantially hybridises to the polynucleotide sequence of the present invention. Such hybridisation may occur at or between low and high stringency conditions. In general terms, low stringency conditions can be defined a hybridisation in which the washing step takes place in a 0.330-0.825 M NaCl buffer solution at a temperature of about 40-48° C. below the calculated or actual melting temperature (Tₘ) of the probe sequence (for example, about ambient laboratory temperature to about 55° C.), while high stringency conditions involve a wash in a 0.0165-0.0330 M NaCl buffer solution at a temperature of about 5-10° C. below the calculated or actual Tₘ of the probe (for example, about 65° C.). The buffer solution may, for example, be SSC buffer (0.15M NaCl and 0.015M tri-sodium citrate), with the low stringency wash taking place in 3×SSC buffer and the high stringency wash taking place in 0.1×SSC buffer. Steps involved in hybridisation of nucleic acid sequences have been described for example in Sambrook et al. (1989; Molecular Cloning, Cold Spring Harbor Laboratory Press, Cold Spring Harbor).

Typically, variants have 77% or more of the nucleotides in common with the nucleic acid sequence of the present invention, for example 78%, 79%, 80%, 81%, 82%, 83%, 84%, 85%, 86%, 87%, 88%, 89%, 90%, 91%, 92%, 93%, 94%, 95%, 96%, 97%, 98% or 99% or greater sequence identity.

Variant nucleic acids of the invention may be codon-optimised for expression in a particular host cell.

DNA polymerases and nucleic acids of the invention may be prepared synthetically using conventional synthesizers. Alternatively, they may be produced using recombinant DNA technology or isolated from natural sources followed by any chemical modification, if required. In these cases, a nucleic acid encoding the chimeric protein is incorporated into suitable expression vector, which is then used to transform a suitable host cell, such as a prokaryotic cell such as E. coli. The transformed host cells are cultured and the protein isolated therefrom. Vectors, cells and methods of this type form further aspects of the present invention.

Sequence identity between nucleotide and amino acid sequences can be determined by comparing an alignment of the sequences. When an equivalent position in the compared sequences is occupied by the same amino acid or base, then the molecules are identical at that position. Scoring an alignment as a percentage of identity is a function of the number of identical amino acids or bases at positions shared by the compared sequences. When comparing sequences, optimal alignments may require gaps to be introduced into one or more of the sequences to take into consideration possible insertions and deletions in the sequences. Sequence comparison methods may employ gap penalties so that, for the same number of identical molecules in sequences being compared, a sequence alignment with as few gaps as possible, reflecting higher relatedness between the two compared sequences, will achieve a higher score than one with many gaps. Calculation of maximum percent identity involves the production of an optimal alignment, taking into consideration gap penalties.

In addition to the BLASTP computer program mentioned above, further suitable computer programs for carrying out sequence comparisons are widely available in the commercial and public sector. Examples include the MatGat program (Campanella et al., 2003, BMC Bioinformatics 4: 29), the Gap program (Needleman & Wunsch, 1970, J. Mol. Biol. 48: 443-453) and the FASTA program (Altschul et al., 1990, J. Mol. Biol. 215: 403-410). MatGAT v2.03 is freely available from the website “http://bitincka.com/ledion/matgat/” (accessed 12 Mar. 2009) and has also been submitted for public distribution to the Indiana University Biology Archive (IUBIO Archive). Gap and FASTA are available as part of the Accelrys GCG Package Version 11.1 (Accelrys, Cambridge, UK), formerly known as the GCG Wisconsin Package. The FASTA program can alternatively be accessed publicly from the European Bioinformatics Institute (http://www.ebi.ac.uk/fasta, accessed 12 Mar. 2009) and the University of Virginia (http://fasta.biotech.virginia.edu/fasta_www/cgi or http://fasta.bioch.virginia.edu/fasta_www2/fasta_list2.shtml, accessed 12 Mar. 2009). FASTA may be used to search a sequence database with a given sequence or to compare two given sequences (see http://fasta.bioch.virginia.edu/fasta_www/cgi/search_frm2.cgi, accessed 12 Mar. 2009). Typically, default parameters set by the computer programs should be used when comparing sequences. The default parameters may change depending on the type and length of sequences being compared. A sequence comparison using the MatGAT program may use default parameters of Scoring Matrix=Blosum50, First Gap=16, Extending Gap=4 for DNA, and Scoring Matrix=Blosum50, First Gap=12, Extending Gap=2 for protein. A comparison using the FASTA program may use default parameters of Ktup=2, Scoring matrix=Blosum50, gap=−10 and ext=−2.

In one aspect of the invention, sequence identity is determined using the MatGAT program v2.03 using default parameters as noted above.

As used herein, a “DNA polymerase” refers to any enzyme that catalyzes polynucleotide synthesis by addition of nucleotide units to a nucleotide chain using a nucleic acid such as DNA as a template. The term includes any variants and recombinant functional derivatives of naturally occurring nucleic acid polymerases, whether derived by genetic modification or chemical modification or other methods known in the art.

As used herein, “thermostable” DNA polymerase activity means DNA polymerase activity which is relatively stable to heat and functions at high temperatures, for example 45-100° C., preferably 55-100° C., 65-100° C., 75-100° C., 85-100° C. or 95-100° C., as compared, for example, to a non-thermostable form of DNA polymerase.

### BRIEF DESCRIPTION OF FIGURES

Particular non-limiting embodiments of the present invention will now be described with reference to the following Figures, in which:

FIG. 1 is a diagram showing the structure of the pET24d(+)HIS region used in cloning of a Palaeococcus helgesonii DNA polymerase according to a first embodiment of the invention;

FIG. 2 is an SDS PAGE gel of fractionated expressed Palaeococcus helgesonii DNA polymerase according to the first embodiment of the invention referred to in FIG. 1. Lane M is a Bio-Rad Precision Plus Protein Standard; lane 1 is induced negative control (equivalent of 100 μl E. coli); lane 2 is induced P. helgesonii DNA polymerase-containing clone (equivalent of 50 μl E. coli); lane 3 is induced HIS-tagged P. helgesonii DNA polymerase-containing clone (equivalent of 50 μl E. coli); lane 4 is induced P. helgesonii DNA polymerase-containing clone (equivalent of 12.5 μl E. coli); lane 5 is induced HIS-tagged P. helgesonii DNA polymerase-containing clone (equivalent of 12.5 μl E. coli); lane 6 is induced P. helgesonii DNA polymerase-containing clone (equivalent of 5 μl E. coli); lane 7 is induced HIS-tagged P. helgesonii DNA polymerase-containing clone (equivalent of 5 μl E. coli); and lane 8 is 25 u Pfu polymerase; and

FIG. 3 is an agarose gel of fractionated PCR reaction samples following amplification of lambda (λ) DNA using the Palaeococcus helgesonii DNA polymerase according to the first embodiment of the invention referred to in FIGS. 1 and 2. Lane M is an EcoR I/Hind III Lambda DNA marker (band sizes (in bp):564, 831, 947, 1375, 1584, 1904, 2027, 3530, 4268, 4973, 5148, 21226); lane 1 is a PCR sample amplified using 1.25 u Pfu polymerase (positive control); and lane 2 is a PCR sample amplified using 2.5 μl of an E. coli extract of a P. helgesonii DNA polymerase-containing clone (non-HIS tagged).

### EXAMPLES

Lyophilized cultures of Palaeococcus helgesonii were obtained from the Deutsche Sammlung von Mikroorganismen and Zellkulturen GmbH (German Collection of Microorganisms and Cell Cultures; Accession No. DSM 15127). As described below, following extraction and amplification of gDNA from the cultures, a gene walking method was used, as outlined below, to reach the predicted 5′ start and the 3′ stop of a putative DNA polymerase B gene (“DNA polB”) encoding a putative DNA polymerase II.

### Example 1

### Genomic DNA Extraction

The method for genomic DNA extraction from P. helgesonii cultures was derived from Gotz et al. (2002; Int. J. Syst. Evol. Microbiol. 52: 1349-1359) which is a modification of a method described in Ausubel et al. (1994; Current Protocols in Molecular Biology, Wiley, New York).

Cell pellets were resuspended in 567 μl 1×TE buffer (10 mM Tris/HCl, pH8.0; 1 mM EDTA), 7.5% Chelex 100 (Sigma), 50 mM EDTA (pH7.0), 1% (w/v) SDS and 200 μg Proteinase K and incubated with slow rotation for 1 h at 50° C. Chelex was removed by centrifugation. Then 100 μl 5M NaCl and 80 μl 10% (w/v) cetyltrimethylammonium bromide in 0.7M NaCl were added to the cell lysate and the sample incubated for 30 mins at 65° C. The DNA was extracted with phenol/chloroform, isopropanol precipitated and the DNA resuspended in water. DNA concentration was estimated on a 1% agarose gel.

### Example 2

### Initial Screening for Putative DNA polB Gene

The screening method was derived from Shandilya et al. (2004, Extremophiles 8: 243-251) and Griffiths et al. (2007, Protein Expression & Purification 52:19-30).

Using degenerate Pol primers ARCHPOLR1 and ARCHPOLF1 (see below), a ˜730 bp fragment was amplified from 10 ng P. helgesonii gDNA.

The ARCHPOLR1 primer has the sequence:

(SEQ ID NO: 3)
5′-CGC GGG AGA ACC TGG TTN TCD ATR TAR TA-3′

(corresponding to the amino acid sequence YYIENQVLP, SEQ ID NO:4); and
the ARCHPOLF1 primer has the sequence:

(SEQ ID NO: 5)
5′-TAC TAC GGA TAG GCC AAR GCN AGR TGG TA-3′

(corresponding to the amino acid sequence YYGXANARW, SEQ ID NO:6).

“X” in SEQ ID NO:6 represents a “STOP” codon, as derived from the primer sequence which is as used by Griffiths et al. The primer is still effective in this gene walking method as demonstrated in the present application and also by the work of Griffiths et al.

The PCR reaction mix was as follows:

| | | |
|---|---|---|
| 10x PCR Buffer (750 mM Tris-HCl, pH 8.8, | 10 | μl |
| 200 mM (NH₄)₂SO₄, 0.1% (v/v) Tween-20) | | |
| 5 mM dNTP's | 2 | μl |
| 5′ primer (10 pM/μl) | 2.5 | μl |
| 3′ primer (10 pM/μl) | 2.5 | μl |
| gDNA | 10 | ng |
| Taq DNA Polymerase (5 u/μl) | 0.25 | μl |
| Water | To 50 | μl. |

PCR cycling conditions were 4 minute initial denaturation at 94° C. followed by 15 cycles of: 10 seconds denaturation at 94° C., 30 seconds annealing at 60° C. (reducing by 1° C. per cycle), 1 minute extension at 72° C. This was followed by a further step of 35 cycles of: 10 seconds denaturation at 94° C., 10 seconds annealing at 55° C., 1 minute extension at 72° C. Final extension at 72° C. for 7 mins. 4° C. hold.

A ˜730 bp amplified product was TA cloned (Invitrogen pCR2.1 kit. Cat#K2000-01) and sequenced using M13 Forward (5′-TGT AAA ACG ACG GCC AGT-3′, SEQ ID NO:7) and Reverse (5′-AGCGGATAACAATTTCACACAGGA-3′, SEQ ID NO:8) primers on an ABI-3100 DNA sequencer. Sequencing data confirmed the fragment was of a putative PolB gene.

Sequence data were aligned with that of previously published DNA polymerase DNA sequence data (P.wo, P.fu, P.gl, P.spGE23, P.ab, P.spST700, T.on, T.spGE8, T.zi, T.spGT, T.hy, T.th, T.spTY, T.li, T.sp9N7, T.fu) and a new primer (15127_{—}1) was designed.

The 15127_{—}1 primer has the sequence:

| | |
|---|---|
| 5′ - CAT CCA CAG GAC GTC CC - 3′ | (SEQ ID NO: 9) |

(corresponding to the amino acid sequence HPQDVP, SEQ ID NO:10).

A specific lower primer 15127_L1 (5′-TAAACCCGAATCCCTCTGCC-3′, SEQ ID NO:11) was designed and used in PCR with 15127_{—}1 to amplify a ˜1340 bp fragment.

The PCR reaction mix was as follows:

| | | |
|---|---|---|
| 10x PCR Buffer (750 mM Tris-HCl, pH 8.8, | 5 | μl |
| 200 mM (NH₄)₂SO₄, 0.1% (v/v) Tween-20) | | |
| 5 mM dNTP's | 2 | μl |
| 5′ primer (10 pM/μl) | 2.5 | μl |
| 3′ primer (10 pM/μl) | 2.5 | μl |
| gDNA | 10 | ng |
| Taq DNA Polymerase (5 u/μl) | 0.25 | μl |
| Water | To 50 | μl. |

PCR cycling conditions were 4 minute initial denaturation at 94° C. followed by 15 cycles of: 10 seconds denaturation at 94° C., 10 seconds annealing at 60° C. (reducing by 1° C. per cycle), 2 minute extension at 72° C. This was followed by a further step of 35 cycles of: 10 seconds denaturation at 94° C., 10 seconds annealing at 55° C., 2 minute extension at 72° C. Final extension was at 72° C. for 7 mins. 4° C. hold.

A ˜1340 bp amplified product was ExoSAP treated and sequenced using primer 15127_L1, and later 15127_L2 (5′-TTGTGTGCCTCACGACGGGA-3′, SEQ ID NO:12).

### Example 3

### Gene Walking

From the amplification product obtained in Example 2, primers were designed to ‘walk along’ P. helgesonii gDNA to reach the 5′ start (N-terminus of gene product) and 3′ stop (C-terminus of gene product) of the putative DNA polB gene.

10 ng gDNA was digested individually with 5 u of various 6 base pair-cutter restriction endonucleases in 10 μl reaction volume and incubated for 3 h at 37° C. 12 individual digest reactions were run, using a unique 6-cutter restriction enzyme (RE) for each. 5 μl digested template was then self-ligated using 12.5 u T4 DNA Ligase, 1 μl 10× ligase buffer in 50 μl reaction volume, with an overnight incubation at 16° C.

Self-ligated DNA was then used as template in two rounds of PCR, the second of which used nested primers to give specificity to amplification.

### C-Terminus End:

Primers were designed from the ˜730 bp sequenced fragment to ‘walk’ to the end of the DNA polymerase gene.

The primers were:

| | |
|---|---|
| 15127_C-ter_Upper | |
| (5′-GCAAGGGGCATCAAGGTGAAGC-3′) | SEQ ID NO: 13 |
| 15127_C-ter_Upper_Nested | |
| (5′-TGTTTTGAAGGGGAGCAAGAAG-3′) | SEQ ID NO: 14 |
| 15127_C-ter_Lower | |
| (5′-GCTTTTCTACGGGAACGCGGTA-3′) | SEQ ID NO: 15 |
| 15127_C-ter_Lower_Nested | |
| (5′-GTGACGCTCTCGGCACACTC-3′). | SEQ ID NO: 16 |

### First Round PCR:

The PCR reaction mix was as follows:

| | | |
|---|---|---|
| Self-ligation reaction (~100 pg/μl DNA) | 2 | μl |
| 10x PCR Buffer (200 mM Tris-HCl, pH 8.8, | 5 | μl |
| 100 mM KCl, 100 mM (NH₄)₂SO₄, 1% (v/v) | | |
| Triton X-100, 20 mM MgSO₄) | | |
| 5 mM dNTP's | 2 | μl |
| 15127_C-ter_Upper | 25 | pM |
| 15127_C-ter_Lower | 25 | pM |
| Taq/Pfu (20:1) (5 u/μl) | 1.25 | u |
| Water | To 50 | μl. |

Cycling conditions were 4 minute initial denaturation at 94° C. followed by 35 cycles of: 10 seconds denaturation at 94° C., 10 seconds annealing at 55° C., 5 minute extension at 72° C. Final extension was at 72° C. for 7 mins. 4° C. hold.

### Second Round (Nested) PCR:

The PCR reaction mix was as follows:

| | | |
|---|---|---|
| 1^{st} round PCR reaction | 1 | μl |
| 10x PCR Buffer (200 mM Tris-HCl, pH 8.8, | 5 | μl |
| 100 mM KCl, 100 mM (NH₄)₂SO₄, 1% (v/v) | | |
| Triton X-100, 20 mM MgSO₄) | | |
| 5 mM dNTP's | 2 | μl |
| 15127_ C-ter_Upper_Nested | 25 | pM |
| 15127_ C-ter_Lower_Nested | 25 | pM |
| Taq/Pfu (20:1) (5 u/μl) | 1.25 | u |
| Water | To 50 | μl. |

Cycling conditions were 4 minute initial denaturation at 94° C. followed by 25 cycles of: 10 seconds denaturation at 94° C., 10 seconds annealing at 55° C., 5 minute extension at 72° C. Final extension was at 72° C. for 7 mins. 4° C. hold.

PCR fragments between ˜0.5 kb and ˜2.5 kb were obtained from Nco I, Hind III, Nhe I, Fsp I digested/self-ligated reaction templates.

These fragments were sequenced using the nested primers. Sequencing of fragments indicated that the C-terminal STOP codon of the DNA polymerase gene had been reached.

### N-Terminus End:

Primers were designed from the ˜1340 bp sequenced fragment to ‘walk’ to the start of the DNA polymerase gene.

These primers were:

| | |
|---|---|
| 15127_N-ter_Lower_Nested | |
| (5′-CCACAACGGCAGGATGCTTC-3′) | SEQ ID NO: 17 |
| 15127_N-ter_Lower | |
| (5′-TAGATGTCCACAACGGCAGG-3′) | SEQ ID NO: 18 |
| 15127_N-ter_Upper | |
| (5′-CAGAGGGCTTGTTCCGATGG-3′). | SEQ ID NO: 19. |

### First Round PCR:

The PCR reaction mix was as follows:

| | | |
|---|---|---|
| Self-ligation reaction (~100 pg/μl DNA) | 2 | μl |
| 10x PCR Buffer (200 mM Tris-HCl, pH 8.8, | 5 | μl |
| 100 mM KCl, 100 mM (NH₄)₂SO₄, 1% (v/v) | | |
| Triton X-100, 20 mM MgSO₄) | | |
| 5 mM dNTP's | 2 | μl |
| 15127_N-ter_Upper | 25 | pM |
| 15127_N-ter_Lower | 25 | pM |
| Taq/Pfu (20:1) (5 u/μl) | 1.25 | u |
| Water | To 50 | μl. |

Cycling conditions were 4 minute initial denaturation at 94° C. followed by 35 cycles of: 10 seconds denaturation at 94° C., 10 seconds annealing at 55° C., 5 minute extension at 72° C. Final extension was at 72° C. for 7 mins. 4° C. hold.

### Second Round (Nested) PCR:

The PCR reaction mix was as follows:

| | | |
|---|---|---|
| 1^{st} round PCR reaction | 1 | μl |
| 10x PCR Buffer (200 mM Tris-HCl, pH 8.8, | 5 | μl |
| 100 mM KCl, 100 mM (NH₄)₂SO₄, 1% (v/v) | | |
| Triton X-100, 20 mM MgSO₄) | | |
| 5 mM dNTP's | 2 | μl |
| 15127_N-ter_Upper | 25 | pM |
| 15127_N-ter_Lower_Nested | 25 | pM |
| Taq/Pfu (20:1) (5 u/μl) | 1.25 | u |
| Water | To 50 | μl. |

Cycling conditions were 4 minute initial denaturation at 94° C. followed by 25 cycles of: 10 seconds denaturation at 94° C., 10 seconds annealing at 55° C., 5 minute extension at 72° C. Final extension was at 72° C. for 7 mins. 4° C. hold.

PCR fragments between ˜0.5 kb and ˜3.5 kb were obtained from Nco I, Nde I, Nsi I, Xho I digested/self-ligated reaction templates.

These fragments were sequenced using the nested round primers. Sequencing of the fragments showed that the N-terminal ATG start codon had been reached.

### Example 4

### Amplification of DNA Polymerase Gene

The gene walking protocols described in Example 3 reached the predicted start and stop of the DNA polymerase (polB) gene. Specific primers were designed to amplify the ˜2.3 kb full length gene (as determined by alignments with previously reported DNA polymerases such as Pfu).

Restriction sites (underlined) were built into primers to allow easy cloning into vectors.

The primers were:

15127_FL_Start_(NcoI)
(SEQ ID NO: 20)
5′-AAGCTTCCATGGGTATTCTTGATACAGATTATATAACGGA-3′
15127_STOP_(SalI)
(SEQ ID NO: 21)
5′-GGATCCGTCGACTTACCCCTTCCCCGTTCCAAGCCACGC-3′;

Gene products were amplified using a high fidelity Phusion DNA polymerase (New England Biolabs).

The PCR solution consisted of:

| | | |
|---|---|---|
| 5x HF Phusion reaction Buffer | 20 | μl |
| 5 mM dNTP's | 4 | μl |
| 5′ primer [15127_FL_Start_(NcoI)] | 25 | pM |
| 3′ primer [15127_STOP_(SalI)] | 25 | pM |
| gDNA | 10 | ng |
| Phusion DNA Polymerase (2 u/μl) | 0.5 | μl |
| Water | To 100 | μl. |

Cycling conditions were: 30 seconds initial denaturation at 98° C. followed by 25 cycles of: 3 seconds denaturation at 98° C., 10 seconds annealing at 55° C., 2.5 minute extension at 72° C. Final extension was at 72° C. for 7 mins. 4° C. hold.

### Example 5

### pET24d(+)HIS Vector Construction

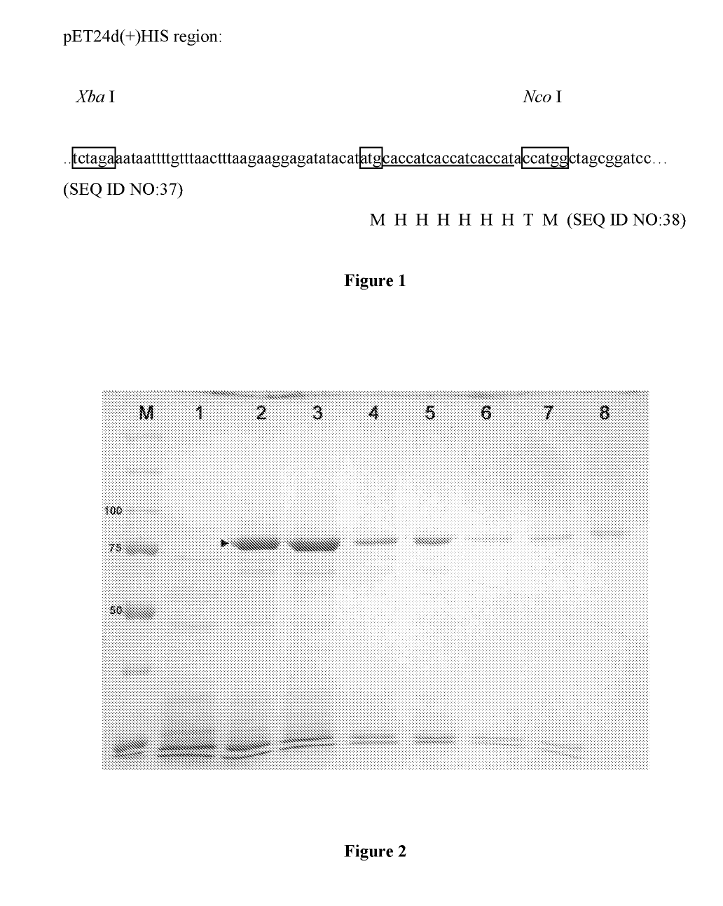

The pET24d(+) vector (Novagen) was modified to add a 6×HIS tag upstream of NcoI site (see FIG. 1). The HIS tag was inserted between XbaI and BamHI sites as follows.

An overlapping primer pair, of which an upper primer (XbaI) has the sequence:

(SEQ ID NO: 22)
5′-TTCCCCTCTAGAAATAATTTTGTTTAACTTTAAGAAGGAGATATA
CATATGCACC-3′

a lower primer (BamHI) has the sequence:

(SEQ ID NO: 23)
5′-GAATTCGGATCCGCTAGCCATGGTATGGTGATGGTGATGGTGCAT
ATGTATATCT-3′

were amplified by PCR, RE digested and ligated into pET24d(+). The ligation reaction was transformed into E. coli TOP10F′ (Invitrogen) and plated on Luria Broth plates plus kanamycin. Colonies were screened by PCR and verified by sequencing using T7 sequencing primers:

| | |
|---|---|
| T7_Promoter: | |
| 5′-AAATTAATACGACTCACTATAGGG-3′ | (SEQ ID NO: 24) |
| T7_Terminator: | |
| 5′-GCTAGTTATTGCTCAGCGG-3′ | (SEQ ID NO: 25) |

### Example 6

### Cloning of DNA Polymerase

The ˜3.9 kb fragment PCR product from Example 4 was purified using Promega Wizard purification kit and then RE digested using Nco I/Sal I. DNA was phenol/chloroform extracted, ethanol-precipitated and resuspended in water. The fragment was then ligated into pET24d(+) and pET24d(+)HIS, between Nco I and Sal I, and electroporated into KRX cells (Promega). Colonies were screened by PCR using vector-specific T7 primers. The KRX (pRARE2) cell strain was produced by electroporating the pRARE2 plasmid (isolated from Rosetta2 [EMD Biosciences]) into E. coli KRX (Promega). The pRARE2 plasmid supplies tRNAs for seven rare codons (AUA, AGG, AGA, CUA, CCC, CGG, and GGA) on a chloramphenicol-resistant plasmid.

### Example 7

### Expression of DNA Polymerase

Recombinant colonies from Example 6 were grown up overnight in 5 ml Luria Broth (including Kanamycin/Chloramphenicol). 50 ml Terrific Broth baffled shake flasks were inoculated by 1/100 dilution of overnight culture. Cultures were grown at 37° C., 275 rpm to OD₆₀₀˜1 then brought down to 24° C. and induced with L-rhamnose to 0.1% final concentration, and IPTG to 10 mM final concentration. Cultures were incubated for a further 18 h at 24° C., 275 rpm. 10 ml of the culture was then harvested by centrifugation for 10 mins at 5,000×g and cells were resuspended in 1 ml Lysis buffer (50 mM Tris-HCl, pH8.0, 100 mM NaCl, 1 mM EDTA) and sonicated for 2 bursts of 30 s (40 v) on ice. Samples were centrifuged at 5,000×g for 5 min and heat lysed at 70° C. for 20 min to denature background E. coli proteins. Samples were centrifuged and aliquots of supernatant were size fractionated on 8% SDS-PAGE.

Expressed protein bands were visible at the expected molecular weight of ˜90 kDa, as shown in FIG. 2.

### Example 8

### PCR Activity Assay

PCR activity of the samples obtained in Example 7 was tested in a 2 kb λDNA PCR assay. Pfu DNA polymerase (1.25 u) was used as positive control.

The PCR solution contained:

| | | |
|---|---|---|
| 10x PCR Buffer (750 mM Tris-HCl, pH 8.8, | 5 | μl |
| 200 mM (NH₄)₂SO₄, 0.1% (v/v) Tween-20) | | |
| 5 mM dNTP mix | 2 | μl |
| Enzyme test sample | 1 | μl |
| Upper λ primer | 25 | pM |
| Lower λ primer | 25 | pM |
| λDNA | 1 | ng |
| Water | To 50 | μl. |

The Upper λ primer has the sequence:

| | |
|---|---|
| 5′ - CCTGCTCTGCCGCTTCACGC - 3′, | (SEQ ID NO: 26) |

while the Lower primer has the sequence:

| | |
|---|---|
| 5′ - CCATGATTCAGTGTGCCCGTCTGG - 3′. | (SEQ ID NO: 27) |

PCR proceeded with 35 cycles of: 3 seconds denaturation at 94° C., 10 seconds annealing at 55° C., 2 minutes extension at 72° C. Final extension at 72° C. for 7 mins. 4° C. hold.

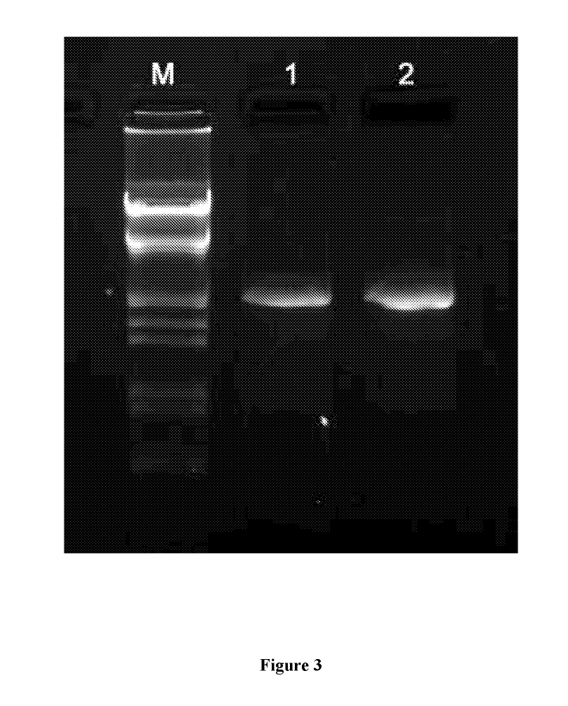

Aliquots of the reaction products were run out on a 1% agarose gel, and the P. helgesonii DNA polymerase was found to amplify the expected 2 kb λ DNA fragment as shown in FIG. 3.

Although the present invention has been described with reference to preferred or exemplary embodiments, those skilled in the art will recognise that various modifications and variations to the same can be accomplished without departing from the spirit and scope of the present invention and that such modifications are clearly contemplated herein. No limitation with respect to the specific embodiments disclosed herein and set forth in the appended claims is intended nor should any be inferred.

All documents cited herein are incorporated by reference in their entirety.

1. A polypeptide having thermostable DNA polymerase activity and comprising or consisting of an amino acid sequence with at least 79% identity to Palaeococcus helgesonii DNA polymerase shown in SEQ ID NO: 1.

2. The polypeptide according to claim 1 comprising or consisting of an amino acid sequence with at least 79% identity to Palaeococcus helgesonii DNA polymerase shown in SEQ ID NO: 39.

3. The polypeptide according to claim 1, which is suitable for carrying out a thermocycling amplification reaction, such as a polymerase chain reaction (PCR).

4. The polypeptide according to claim 1, in which the polypeptide has 3′-5′ exonuclease proofreading activity.

5. The polypeptide according to claim 1, in which the polypeptide lacks 5′-3′ exonuclease activity.

6. The polypeptide according to claim 1, which is an isolated thermostable DNA polymerase obtainable from Palaeococcus helgesonii and having a molecular weight of about 90,000 Daltons, or an enzymatically active fragment thereof.

7. A polypeptide according to claim 1 having thermostable DNA polymerase activity and comprising the amino acid sequence SEQ ID NO: 39.

8. (canceled)

9. A polypeptide according to claim 1, further comprising a Cren7 enhancer domain.

10. A composition comprising the polypeptide of claim 1.

11. The composition according to claim 10, which is enzymatically thermostable.

12. An isolated nucleic acid encoding the polypeptide of claim 1.

13. (canceled)

14. (canceled)

15. A vector comprising the nucleic acid of claim 12.

16. A host cell transformed with the nucleic acid of claim 12.

17. A kit comprising the polypeptide of claim 1, together with packaging materials therefor.

18. A method of amplifying a sequence of a target nucleic acid using a thermocycling reaction, comprising the steps of:
(1) contacting the target nucleic acid with the polypeptide of claim 1, and/or the composition of claim 10 or claims 11; and
(2) incubating the target nucleic acid with the polypeptide and/or composition under thermocycling reaction conditions which allow amplification of the target nucleic acid.

19. (canceled)

20. (canceled)

21. A host cell transformed with the vector of claim 15.

22. A kit comprising the composition of claim 10 or claim 11, together with packaging materials therefor.

23. A kit comprising the nucleic acid of 12, together with packaging materials therefor.

24. A kit comprising the vector of claim 15, together with packaging materials therefor.

25. A kit comprising the host cell of claim 16 or claim 21, together with packaging materials therefor.
